(19) United States
(12) Patent Application Publication
(10) Pub. No.: US 2022/0399113 A1
(43) Pub. Date: Dec. 15, 2022

(54) Systems and Methods for a Virtual, Intelligent and Customizable Personal Medical and Security Assistant

(71) Applicant: Electronic Caregiver, Inc., Las Cruces, MN (US)

(72) Inventors: Anthony Dohrmann, El Paso, TX (US); Bryan John Chasko, Las Cruces, MN (US); Judah Tveito, Las Cruces, MN (US); David W. Keeley, Frisco, TX (US)

(21) Appl. No.: 17/893,982

(22) Filed: Aug. 23, 2022

Related U.S. Application Data
(63) Continuation-in-part of application No. 16/416,062, filed on May 17, 2019, now Pat. No. 11,488,724.

(51) Int. Cl. G16H 40/67; G16H 10/60; G10L 15/22; G10L 15/08; G06T 19/00

(52) U.S. Cl. CPC G16H 40/67; G16H 10/60; G10L 15/22; G10L 15/08; G06T 19/006; G10L 2015/088

(57) ABSTRACT

Embodiments of the present technology pertain to methods and systems for a voice based, intelligent, augmented reality (AR) based on-demand medical assistant receiving health data of a user using medical testing equipment. Embodiments comprise: storing the health data of the user in a retrieval database; receiving an audio input using a web-browser based AR, animated, conversational graphical user interface, the audio input comprising keywords; determining a domain of use based on processing the keywords of the audio input; processing the health data of the user, the keywords, and the domain of use to determine a medical assessment for the user; determining personalized medical services for the user based on the medical assessment; providing the user with access to the personalized medical services using the web-browser based AR, animated, conversational graphical user interface; and displaying a status of the personalized medical services to the user using the graphical user interface.

### CROSS REFERENCE TO RELATED APPLICATIONS

This continuation in part application claims the priority benefit of U.S. Non-Provisional patent application Ser. No. 16/416,062 filed on May 17, 2019 and titled “Systems and Methods for a Virtual, Intelligent and Customizable Personal Medical Assistant,” which claims the priority benefit of U.S. Provisional Patent Application Ser. No. 62/686,325 filed on Jun. 18, 2018 and titled “Virtual, Intelligent and Customizable Personal Medical Assistant,” all of which are hereby incorporated by reference in their entireties.

### FIELD OF THE TECHNOLOGY

Embodiments of the present technology relate to systems and methods for a voice based, intelligent, augmented reality (AR) based on-demand medical assistant.

### SUMMARY

According to some embodiments, the present technology is directed to methods for a voice based, intelligent, augmented reality (AR) based on-demand medical assistant. In some embodiments the method comprises: (a) receiving health data of a user using medical testing equipment; (b) storing the health data of the user in a retrieval database; (c) receiving an audio input using a web-browser based AR, animated, conversational graphical user interface, the audio input comprising keywords; (d) determining a domain of use based on processing the keywords of the audio input; (e) processing the health data of the user, the keywords, and the domain of use to determine a medical assessment for the user; (f) determining personalized medical services for the user based on the medical assessment; (g) providing the user with access to the personalized medical services using the web-browser based AR, animated, conversational graphical user interface; and (h) displaying a status of the personalized medical services to the user using the web-browser based AR, animated, conversational graphical user interface.

In some embodiments of the present technology, the methods further comprise actively prompting the user for health data using the web-browser based AR, animated, conversational graphical user interface.

In some embodiments the processing the health data of the user, the keywords, and the domain of use utilizes a medical and health vocabulary database.

In various embodiments the processing the health data of the user, the keywords, and the domain of use utilizes a secure cloud-based repository of user provided data, a public domain medical and health database, and a public domain informational repository.

In some embodiments the health data of a user comprises a baseline health level of the user.

In some embodiments of the present technology, the methods further comprise determining when the baseline health level of the user deviates from a predetermined threshold.

In some embodiments of the present technology, the methods further comprise displaying an alert to the user when the baseline health level of the user changes past a threshold via the web-browser based AR, animated, conversational graphical user interface.

In various embodiments of the present technology, the methods further comprise sending an alert to a third-party medical service provider when the baseline health level of the user deviates from the predetermined threshold.

In some embodiments the health data of the user comprises a disease profile of the user.

In various embodiments of the present technology, the methods further comprise providing the user with a treatment plan specific the disease profile of the user using the web-browser based AR, animated, conversational graphical user interface.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure, and explain various principles and advantages of those embodiments.

FIG. 1 shows a diagram of system architecture for an exemplary system configured to provide a voice based, intelligent, augmented reality (AR) based on-demand medical assistant according to embodiments of the present technology.

FIG. 2 shows devices for a voice based, intelligent, augmented reality (AR) based on-demand medical assistant according to embodiments of the present technology.

FIG. 3 shows embodiments of a voice based, intelligent, augmented reality (AR) based on-demand medical assistant displayed in a home according to embodiments of the present technology.

FIG. 4 shows keyword identification functionality according to embodiments of the present technology.

FIG. 5 illustrates exemplary graphical user interfaces for general medication requirements using a voice based, intelligent, augmented reality (AR) based on-demand medical assistant according to embodiments of the present technology.

FIG. 6A and FIG. 6B illustrate exemplary graphical user interfaces for provision of emergency medical actions using a voice based, intelligent, augmented reality (AR) based on-demand medical assistant according to embodiments of the present technology.

FIG. 7 shows a system for designating a plan of care using a voice based, intelligent, augmented reality (AR) based on-demand medical assistant according to embodiments of the present technology.

FIG. 8 shows a system for third-party contact protocol resulting from real-time data analysis using a voice based, intelligent, augmented reality (AR) based on-demand medical assistant displayed according to embodiments of the present technology.

FIG. 9 illustrates exemplary graphical user interfaces for provision of emergency medical actions using a voice based, intelligent, augmented reality (AR) based on-demand medical assistant according to embodiments of the present technology.

FIG. 10 illustrates an exemplary computer system that may be used for a voice based, intelligent, augmented reality (AR) based on-demand medical assistant according to embodiments of the present technology.

### DETAILED DESCRIPTION

In the following description, for purposes of explanation, numerous specific details are set forth in order to provide a thorough understanding of the disclosure. It will be apparent, however, to one skilled in the art, that the disclosure may be practiced without these specific details. In other instances, structures and devices may be shown in block diagram form only in order to avoid obscuring the disclosure. It should be understood, that the disclosed embodiments are merely exemplary of the invention, which may be embodied in multiple forms. Those details disclosed herein are not to be interpreted in any form as limiting, but as the basis for the claims.

According to the embodiments of the present technology, an automated, voice-based and intelligent AR virtual medical assistant is by way of an electronic device. This medical assistant facilitates user interaction with the device to assist the user with various home healthcare activities. The medical assistant is integrated into the environment of the user and utilizes conversational platforms to achieve the following: access to various external services, providing reminders associated with various user medical needs, obtaining relevant medical information associated with the user, and providing appropriate feedback based on various medical information collected.

In various embodiments the present technology provides a user with continuous access to a virtual care provider via interaction with connected devices of the user. Embodiments of the present technology function to provide quick responses to a variety of health care related inquiries and tasks and relay requested and necessary information/materials to individuals via the display of a user's connected devices. Additionally, the capability of having Internet or cellular network connectivity allows the virtual health assistant to connect users with 24/7 telehealth service, 24/7 emergency monitoring and dispatch capabilities, monitored medication and medical test reminders and provide immediate response to failures associated with user provided medication compliance responses. The addition of ancillary hardware provides the capability for the virtual health assistant to assess and interpret user activity levels, respond to inactivity and falls, deliver questionnaire type health assessments, track symptoms and monitor for potential changes in condition. For example, these capabilities allow the virtual health assistant to effectively provide methods and systems for continued care and monitoring for the user.

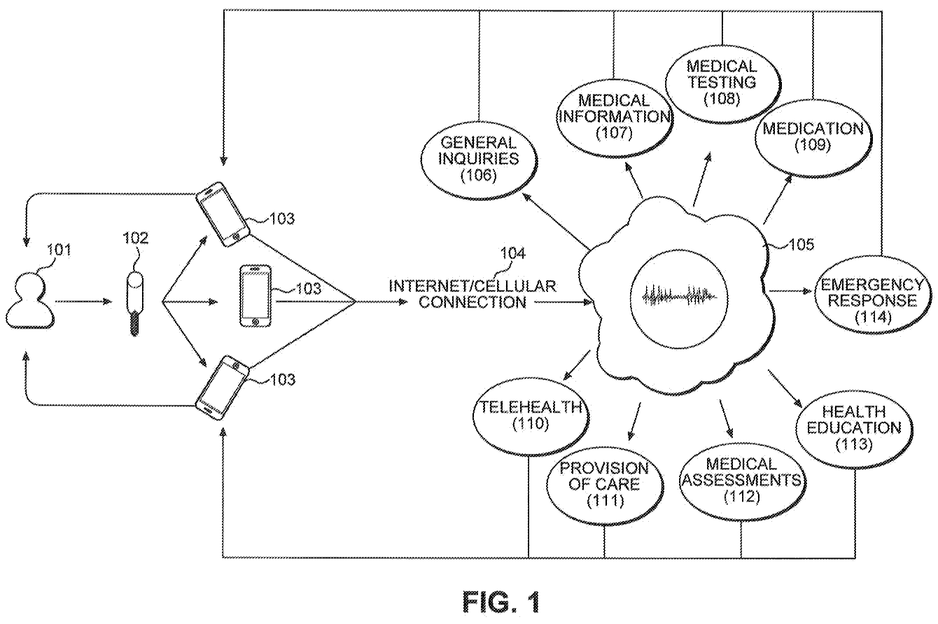

FIG. 1 shows a diagram of system architecture for an exemplary system configured to provide a voice based, intelligent, augmented reality (AR) based on-demand medical assistant according to embodiments of the present technology. FIG. 1 shows an augmented reality based virtual health assistant system. As such, the user 101 utilizes the mechanism for voice input 102 to communicate with a connected device 103 (e.g., mobile device). The connected device 103 is connected to a network. For example, an internet/cellular connection 104. The network (e.g., Internet/cellular connection 104) may include a wireless or wire network, or a combination thereof. For example, the network may include one or more of the following: the Internet, local intranet, PAN (Personal Area Network), LAN (Local Area Network), WAN (Wide Area Network), MAN (Metropolitan Area Network), virtual private network (VPN), storage area network (SAN), frame relay connection, Advanced Intelligent Network (AIN) connection, synchronous optical network (SONET) connection, digital T1, T3, E1 or E3 line, Digital Data Service (DDS) connection, DSL (Digital Subscriber Line) connection, Ethernet connection, ISDN (Integrated Services Digital Network) line, dial-up port such as a V.90, V.34 or V.34bis analog modem connection, cable modem, ATM (Asynchronous Transfer Mode) connection, or an FDDI (Fiber Distributed Data Interface) or CDDI (Copper Distributed Data Interface) connection. Furthermore, the communications may also include links to any of a variety of wireless networks including, WAP (Wireless Application Protocol), GPRS (General Packet Radio Service), GSM (Global System for Mobile Communication), CDMA (Code Division Multiple Access) or TDMA (Time Division Multiple Access), cellular phone networks, GPS, CDPD (cellular digital packet data), RIM (Research in Motion, Limited) duplex paging network, Bluetooth radio, or an IEEE 802.11-based radio frequency network. The network can further include or interface with any one or more of the following: RS-232 serial connection, IEEE-1394 (Firewire) connection, Fiber Channel connection, IrDA (infrared) port, SCSI (Small Computer Systems Interface) connection, USB (Universal Serial Bus) connection, or other wired or wireless, digital or analog interface or connection, mesh or Digi® networking.

In various embodiments of the present technology the internet/cellular connection 104 allows voice input from user 101 to be analyzed by a cloud-based keyword processor 105 and directed to an appropriate domain of inquiry. The domains of inquiry associated with the system may include, but are not limited to areas such as general inquiries 106, medical information 107, medical testing 108, medication 109, telehealth 110, provision of care 111, medical assessments 112, health education 113 and emergency response services 114. Following direction to the appropriate domain of inquiry, cloud-based compute power of the present technology executes the query submitted by user 101 and the appropriate service output is provided to user 101 using the connected device 103.

Various embodiments of the present technology are available across a wide variety of devices with voice input technology, Internet and/or cellular connectivity, and web-browser capabilities. Systems of the present technology may be implemented with many different types of devices and operational modes. One of ordinary skill in the art associated with the present technology understands that the devices (e.g., connected device 103) and operational modes indicated above are simply examples and are not intended to be exhaustive. As such, it is understood that the present technology may be implemented across additional media meeting the necessary requirements and deployed across a variety of operational modes.

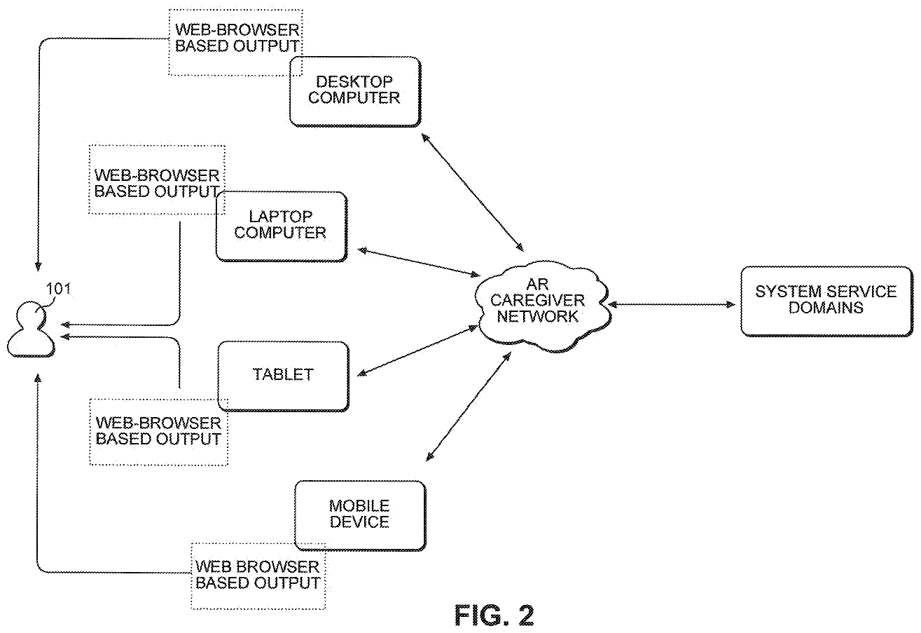

FIG. 2 shows devices for a voice based, intelligent, augmented reality (AR) based on-demand medical assistant according to embodiments of the present technology. FIG. 2 shows a block diagram depicting a system capable of being available on a number of different device types with a web-browser based output. For example, the user 101 may access a desktop computer, a laptop computer, a tablet, and a mobile device that are connected to an AR caregiver network and system service domains.

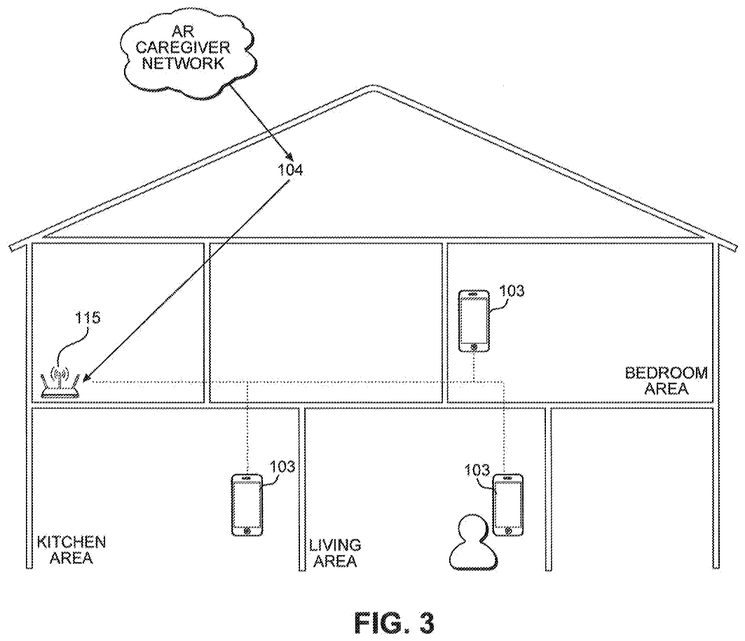

FIG. 3 shows embodiments of a voice based, intelligent, augmented reality (AR) based on-demand medical assistant displayed in a home according to embodiments of the present technology. FIG. 3 shows a representation of systems of the present technology functioning across the operational mode utilized in a household setting. In a household setting operational mode, multiple devices can be distributed throughout the house while connected to a router 115 connected to the Internet/cellular connection 104 (e.g., a network). In addition, systems of the present technology have capacity to function while user 101 is in transit by way of connection only to a cellular network. Systems of the present technology are capable of providing the user 101 with constant access to the variety of services according to various embodiments.

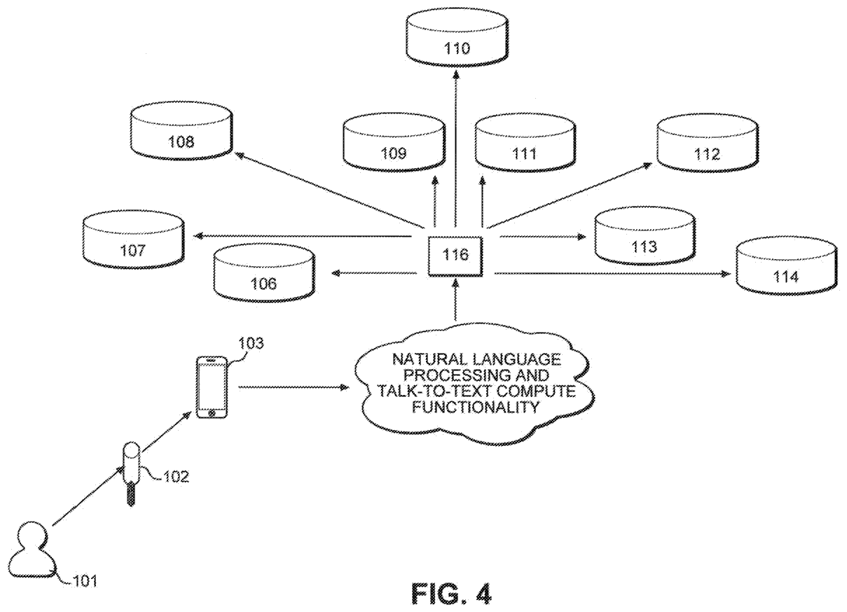

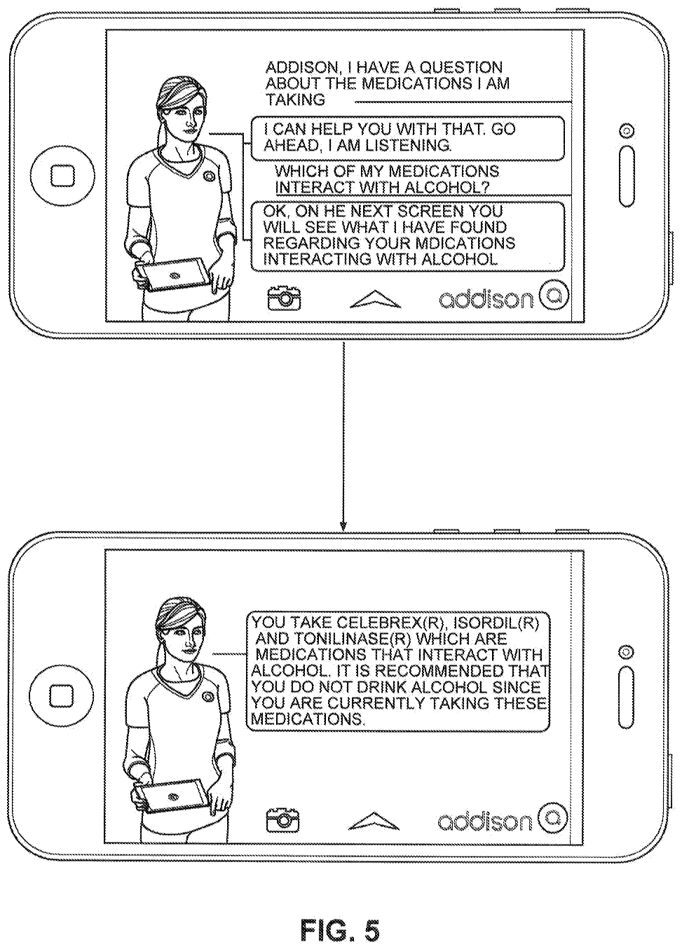

FIG. 4 shows keyword identification functionality according to embodiments of the present technology. FIG. 4 shows embodiments of the present technology are capable of processing voice input data from user 101 such that keywords are identified. Keyword identification from voice input allows the intelligent medical assistant system to quickly access a domain of service desired by user 101 and to carry out a requested function. As such, when the user 101 initiates interactions with systems of the present technology, cloud-based speech-to-text technology converts the user input to text, with the text then being compared to pre-defined keywords stored in a keyword database 116. Upon system recognition of a keyword, the content domain associated with the request of user 101 is accessed. An example is described in FIG. 5. The domains of inquiry associated with the keyword database 116 may include, but are not limited to areas such as general inquiries 106, medical information 107, medical testing 108, medication 109, telehealth 110, provision of care 111, medical assessments 112, health education 113 and emergency response services 114. Following direction to the appropriate domain of inquiry, cloud-based compute power of the present technology executes the query submitted by user 101 and the appropriate service output is provided to user 101 using the connected device 103.

FIG. 5 illustrates exemplary graphical user interfaces for general medication requirements using a voice based, intelligent, augmented reality (AR) based on-demand medical assistant according to embodiments of the present technology. FIG. 5 shows an example of the user 101 interacting with the system described in FIG. 4. For example, the user 101 initiates interaction with the system by stating the audio input “Addison, I have a question about a medication I am taking.” Following this statement by the user 101, the system will process, transmit, and analyze the statement against the various pre-defined keywords stored in the keyword database 116. Upon identifying the pre-defined keyword “medication”, the pathway within the system associated with medication data of the user 101 will be activated and the user 101 will be prompted to begin their medication query. The user 101 will then state a query (i.e., audio input) such as “Addison, which of my medications interact with alcohol?”. Following this query (i.e., audio input), activation of the medication interaction application programming interface (API) call occurs in a manner resulting in a drug-alcohol interaction search that is specific to those medications consumed by the user 101. Following the drug-alcohol interaction search, the results are provided back to the user in via a web-browser based augmented reality (AR) platform and conversational interface displayed in FIG. 5.

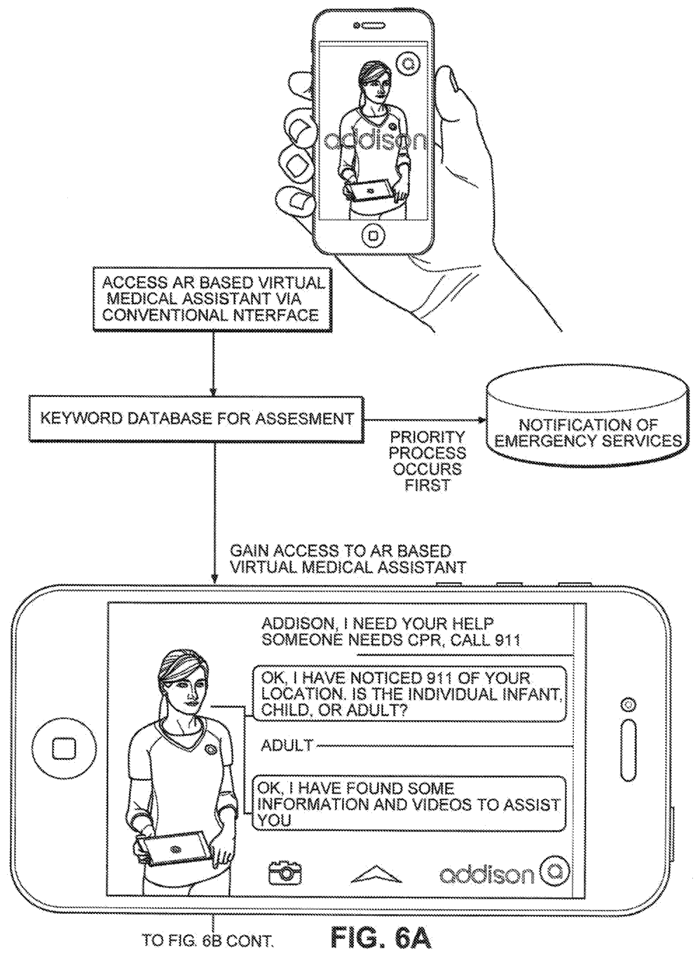

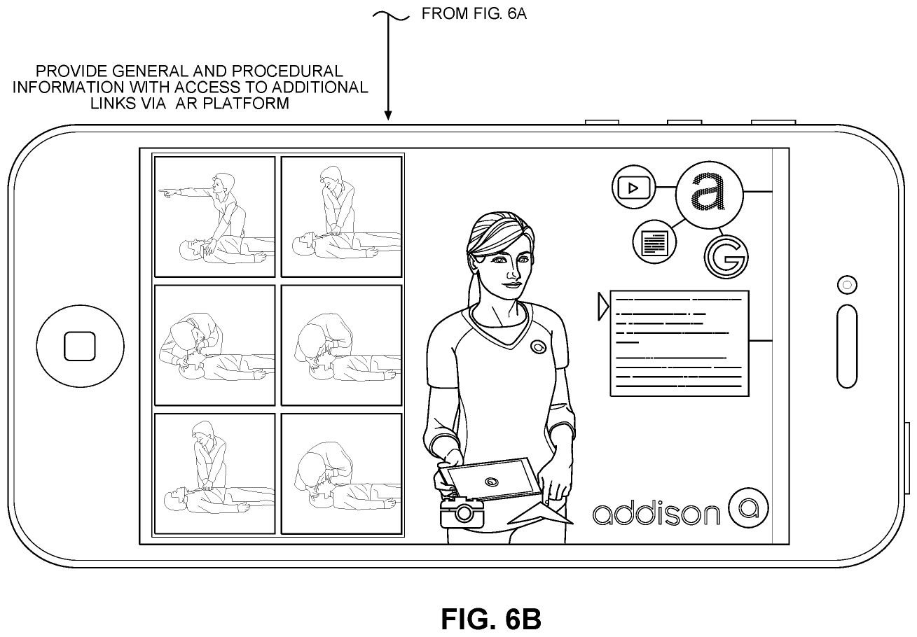

FIG. 6A and FIG. 6B illustrate exemplary graphical user interfaces for provision of emergency medical actions using a voice based, intelligent, augmented reality (AR) based on-demand medical assistant according to embodiments of the present technology. For example, FIG. 6A and FIG. 6B show access notification of emergency services based on interaction of the user 101 with embodiments of the present technology.

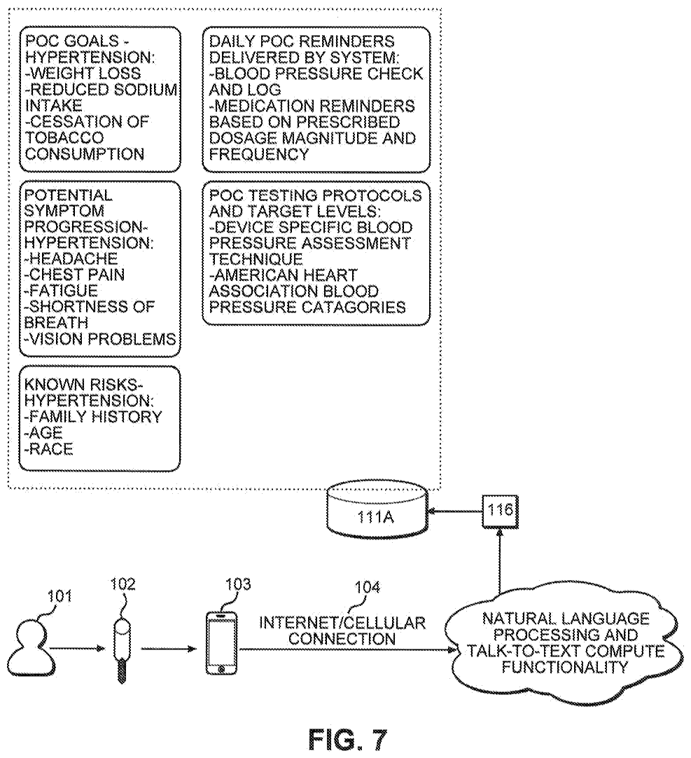

FIG. 7 shows a system for designating a plan of care using a voice based, intelligent, augmented reality (AR) based on-demand medical assistant according to embodiments of the present technology. FIG. 7 shows capacity of the present technology to provide the user 101 with a plan of care (POC) specific to their disease profile. For example, the present technology allows systems to assist individuals diagnosed with chronic medical problems to manage their disease process, yet still remain independent as much as possible. In order for these POCs to be successfully carried out, the user 101 provides basic information regarding their disease profile. As shown FIG. 7, the user 101 establishes their disease profile within the system through use of the conversational interface. After voice input processing and keyword identification by the keyword database 116, a data pathway to a medical database 111A is opened that sets in motion the development of a defined POC for the user 101. This POC is inclusive of: goals the user 101 should meet, basic reminders set to the specific POC, monitoring protocols associated with the specific disease profile, symptoms associated with disease progression, as well as medical testing needs, protocols, frequency, and targets.

In various embodiments of the present technology utilization of a web-browser based AR platform and conversational interface provides the user 101 with the opportunity to interact with the present technology in a manner that allows the system to take in data describing symptoms the user 101 is experiencing in real-time, compare those symptoms with known disease risks, and advise a course of action.

Embodiments of the present technology provide capacity for interacting with additional medical testing devices to take in, store, and assess various biomedical data. As such, the user 101 may be provided consistent feedback related to their disease status and to be guided through disease specific interventions that assist the user 101 in the management of their disease.

In various embodiments of the present technology, implementation of a POC specific to a user with diabetes is provided as an example. Following the POC establishment, the system interacts with the user 101 throughout the course of the day based on the defined characteristics of the POC. In this example, the user 101 has been diagnosed with diabetes and has set-up a POC based on their known risks and management goals. With the user 101 having the known risk factors of a lack of dietary adherence, inadequate blood glucose level monitoring, and a history of medication mismanagement, the system functions to deliver the POC: prompting the user 101 for nutritional planning for items that are appropriate for diabetics, consistently querying user 101 as to the last time of blood glucose testing, and medication management suggestion based on blood glucose test results.

In various embodiments of present technology systems provide the capacity to deliver health provider designated medical questionnaires and assessments to the user 101 without the need for a visit to the practitioner's office. Within the system, the user 101 has the ability to identify Health Insurance Portability and Accountability Act of 1996 (HIPPA) compliant third parties that can provide assessments for completion by the user 101. Once HIPPA compliant third parties are provided access by the user 101, the HIPPA compliant third parties have the capacity to request various medical assessments be delivered to the user 101. For this to occur, the HIPPA compliant third parties access a virtual assistant network via a network that indicates which follow-up/ancillary assessment(s) should be completed by the user 101. The system then notifies the user 101 of the prescribed assessment. Once notified the user 101 communicates with the system via the conversational interface and establishes a date and time to complete the prescribed assessment. Upon completion of the prescribed assessment by the user 101, the data are uploaded to cloud-based storage and can be accesses for evaluation by the prescribing practitioner. For example, the user 101 may be discharged from a hospital following total knee arthroplasty. Following discharge, HIPPA compliant third parties access a virtual assistant network via a connected device and request the user 101 to complete both the Oxford Knee Score questionnaire and/or the Physical Functional Ability Questionnaire. Additionally, a second HIPPA compliant third party requests the user 101 to provide general follow-up information related to how many times the user 101 walks per day, how far the user 101 can walk on average before needing to stop, and how often the user 101 consumes opioid based pain medication. These follow-up inputs are then transmitted to the user 101 via the system and after receipt of request, the user 101 completes the assessments in their home setting by way of the conversational interface and web-browser based AR platform. Once the requested follow-up assessments have been complete, the system transmits the data back to HIPPA compliant third parties and a virtual assistant network for assessment and evaluation.

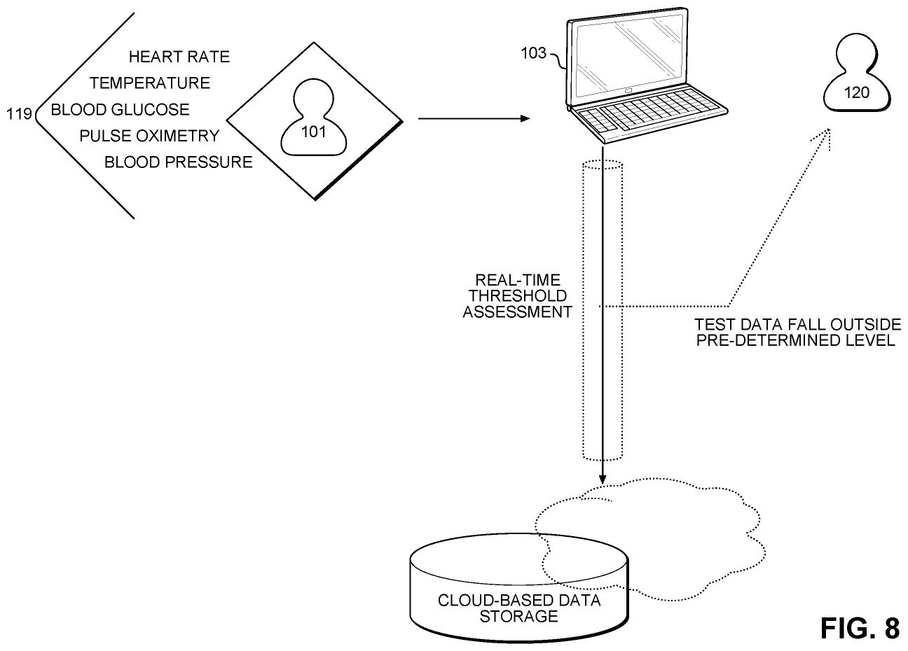

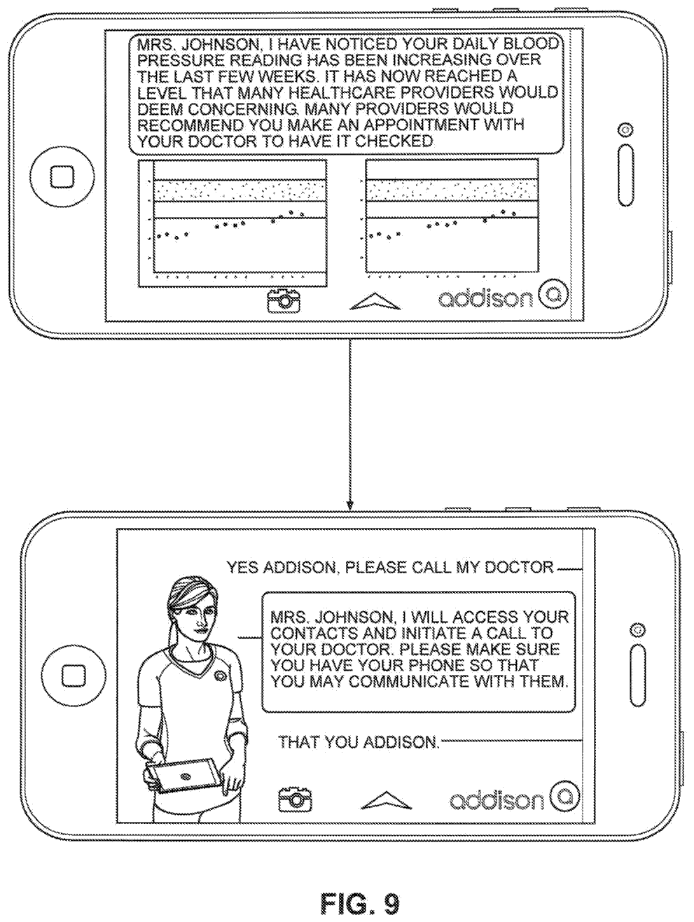

FIG. 8 shows a system for third-party contact protocol resulting from real-time data analysis using a voice based, intelligent, augmented reality (AR) based on-demand medical assistant displayed according to embodiments of the present technology. FIG. 8 shows the ability to provide the user 101 with specific thresholds related to medical testing results that when exceeded, give rise to a notification of a provided third-party contact. As displayed in FIG. 8, a baseline threshold is set for specific characteristics of the disease profile associated with the user 101. Throughout the course of everyday life, the system prompts the user 101 to use the appropriate peripheral medical testing devices 119 to complete various medical tests prescribed as a result of their diagnosis. After testing is completed, these data are transmitted to and stored in a cloud-based database for longitudinal analysis specific to the user 101. During the transmission process, data is analyzed relative to a defined threshold in real-time. Regardless of threshold assessment results, the medical test data are transmitted to a cloud-based storage instance; however, when threshold analysis identifies medical test data that fall outside of the defined threshold, an immediate notification is transmitted to an indicated third-party contact 120. An example is shown in FIG. 9.

FIG. 9 illustrates exemplary graphical user interfaces for provision of emergency medical actions using a voice based, intelligent, augmented reality (AR) based on-demand medical assistant according to embodiments of the present technology. FIG. 9 shows an assessment of pulse oximetry in cases of Chronic Obstructive Pulmonary Disease (COPD) and/or Congestive Heart Failure (CHF). Although in most cases, pulse oximetry levels between 95% and 100% are considered normal, in both COPD and CHF patients typically present with lower pulse oximetry levels that continue to decline as the disease progresses. By utilizing the system to consistently monitor, store and analyze pulse oximetry levels in real-time, progression of the disease can be remotely monitored. When pulse oximetry levels fall below the set threshold (e.g. 92%) the third-party notification protocol is activated allowing the defined contact (e.g., third-party contact 120) to be notified on a potential exacerbation.

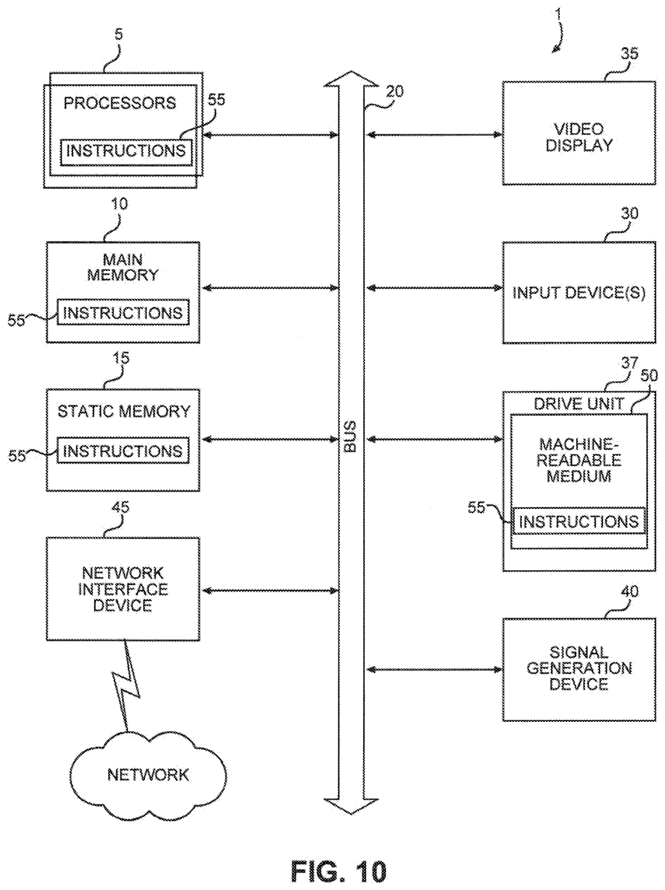

FIG. 10 illustrates an exemplary computer system that may be used for a voice based, intelligent, augmented reality (AR) based on-demand medical assistant according to embodiments of the present technology. FIG. 10 is a diagrammatic representation of an example machine in the form of a computer system 1, within which a set of instructions for causing the machine to perform any one or more of the methodologies discussed herein may be executed. In various example embodiments, the machine operates as a standalone device or may be connected (e.g., networked) to other machines. In a networked deployment, the machine may operate in the capacity of a server or a client machine in a server-client network environment, or as a peer machine in a peer-to-peer (or distributed) network environment. The machine may be a personal computer (PC), a tablet PC, a set-top box (STB), a personal digital assistant (PDA), a cellular telephone, a portable music player (e.g., a portable hard drive audio device such as an Moving Picture Experts Group Audio Layer 3 (MP3) player), a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 1 includes a processor or multiple processor(s) 5 (e.g., a central processing unit (CPU), a graphics processing unit (GPU), or both), and a main memory 10 and static memory 15, which communicate with each other via a bus 20. The computer system 1 may further include a video display 35 (e.g., a liquid crystal display (LCD)). The computer system 1 may also include an alpha-numeric input device(s) 30 (e.g., a keyboard), a cursor control device (e.g., a mouse), a voice recognition or biometric verification unit (not shown), a drive unit 37 (also referred to as disk drive unit), a signal generation device 40 (e.g., a speaker), and a network interface device 45. The computer system 1 may further include a data encryption module (not shown) to encrypt data.

The disk drive unit 37 includes a computer or machine-readable medium 50 on which is stored one or more sets of instructions and data structures (e.g., instructions 55) embodying or utilizing any one or more of the methodologies or functions described herein. The instructions 55 may also reside, completely or at least partially, within the main memory 10 and/or within the processor(s) 5 during execution thereof by the computer system 1. The main memory 10 and the processor(s) 5 may also constitute machine-readable media.

The instructions 55 may further be transmitted or received over a network via the network interface device 45 utilizing any one of a number of well-known transfer protocols (e.g., Hyper Text Transfer Protocol (HTTP)). While the machine-readable medium 50 is shown in an example embodiment to be a single medium, the term “computer-readable medium” should be taken to include a single medium or multiple media (e.g., a centralized or distributed database and/or associated caches and servers) that store the one or more sets of instructions. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media, and carrier wave signals. Such media may also include, without limitation, hard disks, floppy disks, flash memory cards, digital video disks, random access memory (RAM), read only memory (ROM), and the like. The example embodiments described herein may be implemented in an operating environment comprising software installed on a computer, in hardware, or in a combination of software and hardware.

One skilled in the art will recognize that the Internet service may be configured to provide Internet access to one or more computing devices that are coupled to the Internet service, and that the computing devices may include one or more processors, buses, memory devices, display devices, input/output devices, and the like. Furthermore, those skilled in the art may appreciate that the Internet service may be coupled to one or more databases, repositories, servers, and the like, which may be utilized in order to implement any of the embodiments of the disclosure as described herein.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Other exemplary embodiments include a method for a voice based, intelligent, augmented reality (AR) based on-demand medical and security assistant, the method including receiving health data of a user using medical testing equipment, storing the health data of the user in a retrieval database, receiving an audio input using a web-browser based AR, animated, conversational graphical user interface, the audio input comprising keywords, determining a domain of use based on processing the keywords of the audio input, processing the health data of the user, the keywords, and the domain of use to determine a medical assessment for the user, determining personalized medical services for the user based on the medical assessment, providing the user with access to the personalized medical services using the web-browser based AR, animated, conversational graphical user interface, displaying a status of the personalized medical services to the user using the web-browser based AR, animated, conversational graphical user interface and receiving sensor data from the user's environment.

Additionally, according to various exemplary embodiments, the user may be actively prompted for status data using the web-browser based AR, animated, conversational graphical user interface and the sensor data may be from a liquid flow monitor, wherein the sensor data indicates a significant increase in liquid flow and/or the sensor data indicates a significant decrease in liquid flow. The liquid flow monitor may be located in a shower drain, in a bathtub drain and/or the liquid flow monitor may be located in a sink drain. Further exemplary embodiments may include automatically stopping a source of liquid flow and/or contacting the user or a representative of the user about the liquid flow. “Representative of the user” may include a contracted security monitoring center, a contact/triage center and/or other potential sources of contact.

According to various exemplary embodiments, there are many types of flowmeters, and their uses vary. Each flowmeter offers various advantages and capabilities that make it a valuable option in certain instances.

1. Fluid Properties.

Is the fluid a liquid, a gas or steam? How dense or viscous is it? Does it contain any contaminants, such as air bubbles or debris, that could affect the reading? One critical aspect of the fluid to consider is its Reynolds number. This value tells about how a fluid moves through a pipe. If the number is under 2,000, the flow is laminar, meaning it flows steadily. Pay attention to the Reynolds range they are designed to handle, so one can ensure the are a good fit for an application.

Types of Flow Meters.

For example, if one wishes to use an electromagnetic velocity flowmeter, on will need to determine whether a fluid is a conducting substance.

2. Accuracy.

Pay attention to the level of accuracy different flowmeters can deliver. Generally, one of the most accurate types of flowmeters one can find is a positive displacement flowmeter, but this may not always be the best choice, depending on what one is using it for. Accuracy is generally measured in terms of a percentage, such as ±1%. In this example, the reading on the flowmeter could be as much as 1% above or below the actual value. Therefore, the lower the percentage, the more accurate the flowmeter is.

3. Cost-Effectiveness.

Cost is a practical concern that is always worth noting. One may find a flowmeter that would deliver highly accurate results, but is outside the confines of one's budget. Try to balance finding the best possible option with affordability.

Types of Flowmeters.

Exemplary embodiments include many types of flowmeters. Each type goes about measuring the flow rate of a fluid in a different way. Five main types of flowmeters include differential pressure flowmeters, velocity flowmeters, positive displacement flowmeters, mass flowmeters and open-channel flowmeters.

1. Differential Pressure Flowmeters.

One of the most common types of flowmeters is called a differential pressure (DP) flowmeter. This flowmeter type is based on scientific principles that govern the way fluids, both liquids and gases, flow through pipes.

To maintain a consistent flow rate, a fluid that moves from a wider pipe to a narrower one will have to move through with a greater velocity. When a fluid's velocity increases, the pressure decreases.

According to Bernoulli's theorem, one can accurately calculate a fluid's flow rate based on the square root of the differential pressure. In other words, if one can determine the differential pressure, one can determine the flow rate. There are different types of DP flowmeters, but all of them operate according to this principle.

In a differential pressure flowmeter, the fluid you are measuring enters a narrower section of pipe, sometimes by way of an orifice plate. As the name suggests, a differential pressure flowmeter measures the difference in pressure that occurs as a result of the flow-restricted area. Then, a DP flowmeter does the necessary math to display the fluid's flow rate.

Differential flowmeters are common throughout many industries that need to measure the flow rate of fluids. These include the oil and gas, water treatment, pharmaceutical, food and beverage, mining, chemical manufacturing and HVAC industries, among others.

2. Velocity Flow.

Vortex meters include an obstruction that disrupts the fluid's flow and causes it to form vortexes. The number of vortexes formed indicates the fluid's velocity.

Electromagnetic: When other factors remain consistent, the voltage induced by a conducting substance moving across a magnetic field directly relates to the velocity of the fluid. An electromagnetic flowmeter measures the voltage to calculate the flow rate.

Turbine: A turbine meter includes freely rotating propeller blades, positioned perpendicular to the direction the fluid is flowing in. By measuring the frequency of rotations, the turbine flowmeter can calculate the flow velocity.

Paddlewheel: A paddlewheel flowmeter is similar to a turbine meter. In this case, as the paddlewheel turns due to the moving fluid, a sensor picks up on magnets in the paddle as it turns. These produce electrical pulses that indicate the flow rate.

Ultrasonic: Ultrasonic flowmeters use transducers to send sound waves through a fluid. The returning sound wave can signal information about the fluid's flow. This method only works on certain types of fluids.

There are other types of velocity flowmeters, as well. Each is best suited for a particular fluid, but they all manage to calculate velocity to determine flow rate.

3. Positive Displacement

Another common type of flowmeter is the positive displacement (PD) flowmeter. A PD flowmeter is the only type of flowmeter that determines the flow rate of a fluid by measuring the volume of fluid that comes through the meter, rather than measuring some other aspect of the fluid that relates to flow rate and calculating the flow rate based on that.

The meter contains rotating components that resemble mechanical cogs. The movement of the fluid through the meter causes these cogs to rotate. Unlike gears that fit tightly together, these rotating components trap small pockets of fluid in the space between them as they turn. The mechanism counts the rate of rotation of these components to keep track of how much fluid has passed through. This process works because the cogs' rotational velocity links directly to the fluid's flow rate.

The exact way a PD flowmeter measures the rate at which the rotating components turn can vary. One way is for the components to include magnets that trigger sensors outside the fluid chamber.

Positive displacement meters have a reputation for being extremely accurate. This accuracy is partly because their design accounts for nearly all the fluid that passes through. The margin for inaccuracy relates only to the tiny amount of fluid that manages to bypass the seal of the rotating components, sometimes called “slippage.”

In addition to being impressively accurate, PD flowmeters can handle a wide range of fluids when it comes to viscosity. A PD flowmeter may even be able to give a more accurate read for more viscous fluids, since they lend themselves less to slippage.

4. Mass Flow.

A mass flowmeter, as the name suggests, measures the mass flow of fluid as it moves through a pipe. Mass flowmeters are common in the pharmaceutical, mining, wastewater, power and chemical and gas industries. These flowmeters are an ideal choice when one wants to measure mass or when one is working with a very viscous substance.

One popular means of measuring mass flow is by heating a section of fluid and noting the resulting change in temperature. A similar method is to keep a probe at a constant temperature and take note of how much energy the probe requires to do this.

Mass flowmeters that use heat to measure mass flow are called thermal dispersion flowmeters. Another common type of mass flowmeter is a Coriolis mass meter. Regardless of which method one meter uses to measure mass flow rate, one should note there is a difference in mass flow rate and volumetric flow rate.

Whereas a positive displacement flowmeter measures the volume of fluid that passes through, a mass flowmeter measures the mass of the fluid that travels through. A measure of volume would look something like cubic meters per second or another measurement of volume in a given amount of time. Mass is distinct from volume. One may see it measured by kilograms per second or a similar unit of measurement.

By dividing the mass flow rate by the density of the fluid, a mass flowmeter can determine the flow rate in terms of volume. This process sounds fairly straightforward, but it can involve some extra steps to determine the actual density of the fluid, since it may vary depending on conditions.

5. Open Channel.

This type of flowmeter includes a dam-like structure known as a primary device. Typically, the primary device is either a weir or a fume.

If one knows the relationship between depth and flow in pipes, then by placing an obstruction in the pipe, the meter can take note of the difference in depth and use this information to calculate flow rate. The main difference between weirs and flumes is their shape.

Weirs are openings at the top of a dam, which can either be rectangular or V-shaped. As the water or other fluid flows over the dam, through the weir, the height of the fluid will increase. A greater increase in depth indicates a higher flow rate.

Flumes are similar to weirs, but they provide a constriction in width rather than height. As the fluid enters the narrower section of the channel, the water level changes which correlates to the flow rate.

In addition to playing a role in industrial applications such as wastewater treatment, open-channel flowmeters are a useful means of measuring the flow rate in free-flowing bodies of water such as streams and water.

In additional exemplary embodiments, the sensor data is from a thermal sensing device.

Thermal sensors can be classified as:

Thermocouples.

A thermocouple is a non-linear thermal sensor. The sensitivity and temperature ranges of the thermocouple vary with the types of metals bound together. The accuracy of thermocouples is very low, but they offer a broad range of operation, from −200° C. to 1750° C.

Thermocouples are the most commonly-used thermal sensors in industrial, automotive, and consumer applications. They work on the principle of the Seebeck effect—the phenomenon in which the temperature difference between two dissimilar metal wires produces a voltage difference. The voltage difference is proportional to the temperature change. A look-up table is used to convert the voltage difference to temperature measurements.

Resistance Temperature Detectors (RTD).

In RTDs, change in resistance is used for sensing temperature. RTDs require that materials have a well-defined resistance-temperature relationship. Platinum is the best material for RTDs because of its linear relationship between resistance change and temperature variation. Platinum RTDs are stable, accurate, and have repeatability in measurement, with a range of operation from −270° C. to 850° C. Other materials used in RTDs are nickel and copper, but the accuracy is lower with these metals.

Thermistors

Like RTDs, thermistors also make use of resistance in temperature measurements. Thermistors use polymer or ceramic materials instead of platinum and copper, which make them cheaper, but less accurate, than RTDs. There are two types of thermistors:

Negative temperature coefficient (NTC) thermistors—In this type of thermistor, the change in resistance is inversely proportional to the temperature variation.

Positive temperature coefficient (PTC) thermistors—In this type of thermistor, the change in resistance is directly proportional to the temperature variation.

Thermometers.

Thermometers are used to measure the temperature of any glass solids or liquids. Temperature measurements are proportional to the volume change of the temperature sensing element filled inside the thermometer. The accuracy of a thermometer depends on the size of the device and the fluid used as the temperature sensing element.

Infrared (IR) Sensors.

IR sensors are electronic sensors that detect temperature by emitting IR radiations. They are non-contacting type thermal sensors. In IR sensors, the user makes the trade-off between cost and accuracy; the higher the cost of an IR sensor, the higher its accuracy.

Semiconductor Thermal Sensors.

Semiconductor thermal sensors are available as temperature sensor ICs. These ICs detect the temperature variation from the change in output quantities such as current, voltage, resistance, etc. Semiconductor thermal sensor ICs are highly accurate and linear over the temperature range of 55° C. to 155° C.

The sensor data may indicate a significant increase in temperature, and/or the sensor data may indicate a significant decrease in temperature. The thermal sensing device may be located near a stove top, an oven, and/or near a bed. Exemplary embodiments may include automatically stopping a source of heat and/or automatically remotely contacting the user or a representative of the user about temperature. “Representative of the user” may include a contracted security monitoring center, a contact/triage center and/or other potential sources of contact. The health data of a user may include a baseline health level of the user and/or determining when the baseline health level of the user deviates from a predetermined threshold. Additionally, an alert may be displayed to the user when the baseline health level of the user changes past a threshold, via the web-browser based AR, animated, conversational graphical user interface. An alert may be sent to a third-party medical service provider when the baseline health level of the user deviates from the predetermined threshold.

Exemplary systems may include a system for a voice based, intelligent, augmented reality (AR) based on-demand medical and security assistant, the system including medical testing equipment for receiving health data of a user, a retrieval database for storing the health data of the user, a mobile device communicatively coupled to the medical testing equipment, the mobile device including a web-browser based AR, animated, conversational graphical user interface for receiving audio input, the audio input comprising keywords, t least one processor, and a memory storing processor-executable instructions, wherein the at least one processor is configured to implement the following operations upon executing the processor-executable instructions including determining a domain of use based on processing the keywords of the audio input, processing the health data of the user, the keywords, and the domain of use to determine a medical assessment for the user, determining personalized medical services for the user based on the medical assessment, providing the user with access to the personalized medical services using the web-browser based AR, animated, conversational graphical user interface, displaying a status of the personalized medical services to the user using the web-browser based AR, animated, conversational graphical user interface, and receiving sensor data from the user's environment.

In the description, for purposes of explanation and not limitation, specific details are set forth, such as particular embodiments, procedures, techniques, etc. in order to provide a thorough understanding of the present technology. However, it will be apparent to one skilled in the art that the present technology may be practiced in other embodiments that depart from these specific details.

While specific embodiments of, and examples for, the system are described above for illustrative purposes, various equivalent modifications are possible within the scope of the system, as those skilled in the relevant art will recognize. For example, while processes or steps are presented in a given order, alternative embodiments may perform routines having steps in a different order, and some processes or steps may be deleted, moved, added, subdivided, combined, and/or modified to provide alternative or sub-combinations. Each of these processes or steps may be implemented in a variety of different ways. Also, while processes or steps are at times shown as being performed in series, these processes or steps may instead be performed in parallel, or may be performed at different times.

While various embodiments have been described above, it should be understood that they have been presented by way of example only, and not limitation. The descriptions are not intended to limit the scope of the present technology to the particular forms set forth herein. To the contrary, the present descriptions are intended to cover such alternatives, modifications, and equivalents as may be included within the spirit and scope of the present technology as appreciated by one of ordinary skill in the art. Thus, the breadth and scope of a preferred embodiment should not be limited by any of the above-described exemplary embodiments.

Thus, the technology for comprehensive fall risk assessment is disclosed. Although embodiments have been described with reference to specific example embodiments, it will be evident that various modifications and changes can be made to these example embodiments without departing from the broader spirit and scope of the present application. Accordingly, the specification and drawings are to be regarded in an illustrative rather than a restrictive sense.

What is claimed is:

1. A method for a voice based, intelligent, augmented reality (AR) based on-demand medical and security assistant, the method comprising:
receiving health data of a user using medical testing equipment;
storing the health data of the user in a retrieval database;
receiving an audio input using a web-browser based AR, animated, conversational graphical user interface, the audio input comprising keywords;
determining a domain of use based on processing the keywords of the audio input;
processing the health data of the user, the keywords, and the domain of use to determine a medical assessment for the user;
determining personalized medical services for the user based on the medical assessment;
providing the user with access to the personalized medical services using the web-browser based AR, animated, conversational graphical user interface;
displaying a status of the personalized medical services to the user using the web-browser based AR, animated, conversational graphical user interface; and
receiving sensor data from the user's environment.

2. The method as recited in claim 1, further comprising:
actively prompting the user for status data using the web-browser based AR, animated, conversational graphical user interface.

3. The method as recited in claim 1, wherein the sensor data is from a liquid flow monitor.

4. The method as recited in claim 3, wherein the sensor data indicates a significant increase in liquid flow.

5. The method as recited in claim 3, wherein the sensor data indicates a significant decrease in liquid flow.

6. The method as recited in claim 3, wherein the liquid flow monitor is located in a shower drain.

7. The method as recited in claim 3, wherein the liquid flow monitor is located in a bathtub drain.

8. The method as recited in claim 3, wherein the liquid flow monitor is located in a sink drain.

9. The method as recited in claim 3, further comprising automatically stopping a source of liquid flow.

10. The method as recited in claim 3, further comprising remotely contacting the user or a representative of the user about liquid flow.

11. The method as recited in claim 1, wherein the sensor data is from a thermal sensing device.

12. The method as recited in claim 11, wherein the sensor data indicates a significant increase in temperature.

13. The method as recited in claim 11, wherein the sensor data indicates a significant decrease in temperature.

14. The method as recited in claim 11, wherein the thermal sensing device is located near a stove top.

15. The method as recited in claim 11, wherein the thermal sensing device is located near an oven.

16. The method as recited in claim 11, wherein the thermal sensing device is located near a bed.

17. The method as recited in claim 11, further comprising automatically stopping a source of heat.

18. The method as recited in claim 11, further comprising remotely contacting the user or a representative of the user about temperature.

19. The method as recited in claim 1, wherein the health data of a user comprises a baseline health level of the user.

20. The method as recited in claim 19, further comprising:
determining when the baseline health level of the user deviates from a predetermined threshold.

21. The method as recited in claim 20, further comprising:
displaying an alert to the user when the baseline health level of the user changes past a threshold via the web-browser based AR, animated, conversational graphical user interface.

22. The method as recited in claim 20, further comprising:
sending an alert to a third-party medical service provider when the baseline health level of the user deviates from the predetermined threshold.

23. A system for a voice based, intelligent, augmented reality (AR) based on-demand medical and security assistant, the system comprising:
medical testing equipment for receiving health data of a user;
a retrieval database for storing the health data of the user;
a mobile device communicatively coupled to the medical testing equipment, the mobile device comprising:
a web-browser based AR, animated, conversational graphical user interface for receiving audio input, the audio input comprising keywords;
at least one processor; and
a memory storing processor-executable instructions, wherein the at least one processor is configured to implement the following operations upon executing the processor-executable instructions:
determining a domain of use based on processing the keywords of the audio input;
processing the health data of the user, the keywords, and the domain of use to determine a medical assessment for the user;
determining personalized medical services for the user based on the medical assessment;
providing the user with access to the personalized medical services using the web-browser based AR, animated, conversational graphical user interface;
displaying a status of the personalized medical services to the user using the web-browser based AR, animated, conversational graphical user interface; and
receiving sensor data from the user's environment.
